(19) United States
(12) Patent Application Publication
(10) Pub. No.: US 2010/0010524 A1
(43) Pub. Date: Jan. 14, 2010

(54) LIQUID JET SURGICAL INSTRUMENT HAVING A DISTAL END WITH A SELECTIVELY CONTROLLABLE SHAPE

(75) Inventors: James E. Barrington, Lexington, MA (US); Kevin P. Staid, Lowell, MA (US); Derek Bruce Eldridge, North Chelmsford, MA (US)

Correspondence Address: MCCARTER & ENGLISH, LLP BOSTON, 265 Franklin Street, Boston, MA, 02110, US

(73) Assignee: HydroCision, Inc., N. Billerica, MA (US)

(21) Appl. No.: 12/161,884

(22) PCT Filed: Jan. 24, 2007
(86) PCT No.: PCT/US07/02055

§ 371 Date: Aug. 19, 2009

Related U.S. Application Data
(60) Provisional application No. 60/761,448, filed on Jan. 24, 2006.

(51) Int. Cl. A61B 17/3203

(52) U.S. Cl. 606/167

(57) ABSTRACT

The invention provides a variety of surgical instruments for forming a liquid jet, which are useful for performing a wide variety of surgical procedures. In some embodiments, the invention provides surgical liquid jet instruments having a pressure tube and an evacuation tube, where the pressure tube includes at least one nozzle for forming a liquid jet and where the evacuation tube includes a jet-receiving opening for receiving the liquid jet when the instrument is in operation. In some embodiments, the distal ends of both the pressure and evacuation tubes have a first configuration in a non-relaxed state and a second configuration in a more relaxed state. In some embodiments, a straightener is constructed to selectively control the configuration of the distal ends of both the pressure and evacuation tubes. The invention also provides surgical methods utilizing the inventive surgical liquid jet instruments for cutting or ablating a selected tissue within portions of a patient's spine, such as within the intervertebral disc.

### FIELD OF THE INVENTION

The invention relates generally to surgical instruments for creating a liquid jet and methods for using the instruments in surgical procedures.

### BACKGROUND OF THE INVENTION

There has been a trend in recent years to perform many surgical procedures using less invasive techniques by accessing surgical sites via small holes through the skin or through body orifices. These techniques are known as “minimally invasive surgery.” Minimally invasive surgical techniques commonly employed include endoscopic, laparoscopic, and arthroscopic surgical procedures. Minimally invasive surgical procedures are commonly preferred to open surgical procedures for many applications because the minimally invasive procedures induce less trauma to the patient during surgery and involve, in many cases, fewer potential complications and reduced recovery time.

A variety of instruments have been developed and utilized for minimally invasive surgical procedures. Frequently used instruments include blades and scalpel-type instruments, motorized rotary blade instruments, laser instruments, and electrosurgical or electrocautery instruments. Typically, these prior art instruments suffer from a variety of disadvantages. For example, the instruments can be slow and laborious to use, typically they lack the ability to selectively differentiate tissue to be excised from non-target tissue, they tend to have sizes and/or shapes which make access of many surgical sites difficult, and they tend to cause unintended damage to tissue surrounding the intended target tissue. Most prior art instruments also require the operator to manually remove excised tissue, for example with forceps, or require an external source of vacuum to be applied to the surgical site, for example, via an aspiration tube that is separate from the surgical instrument, in order to remove excised tissue. For many minimally invasive surgical applications such as arthroscopy, certain spinal procedures etc., where visualization of the surgical site is typically effected using an imaging system having a probe such as a fiber optic probe inserted into the surgical site, the above mentioned prior art surgical instruments also typically make it difficult to clearly visualize the site of tissue excision within the surgical field by not effectively evacuating tissue and debris from the surgical site. During a minimally invasive procedure, many of the above mentioned prior art surgical instruments are also difficult to maneuver to a desired location once in the body.

Instruments that employ liquid jets have also been utilized in surgical procedures for cutting and ablating tissue. Such instruments have many advantages over the above mentioned surgical instruments for performing both open and minimally invasive surgical procedures. For example, liquid jet instruments can avoid the thermal damage to surrounding tissues that is often caused by instruments such as lasers and electrosurgical devices. In recent years, liquid jet instruments have been utilized for a variety of surgical procedures including open surgical procedures such as liver resection, endoscopic procedures such as kidney stone disruption and removal, and arthrectomy procedures for removal of thrombotic tissue from the vascular system.

A variety of liquid jet instruments for surgery have been developed, including instruments described in commonly-owned U.S. Pat. No.5,944,686, U.S. Pat. No. 6,375,635, U.S. Pat. No. 6,511,493, U.S. Pat. No. 6,451,017, U.S. Pat. No. 7,122,017, U.S. Pat. No. 6,960,182, U.S. Application Publication No. US2003-0125660, U.S. Application Publication No. US2002-0176788, U.S. Application Publication No. US2004-0228736, U.S. Application Publication No. US2004-0243157, U.S. Application Publication No. US2006-0264808, and U.S. Application Publication No. US2006-0229550, which are all incorporated by reference in their entireties.

These surgical liquid jet cutting systems typically have a pump for pressurizing a liquid, such as isotonic saline or other physiologically-compatible liquid. In certain instances, the pressurized liquid is conveyed, for example by flexible tubing, to a handpiece which has a handle region, and a distal end configured to perform a surgical or medical procedure on a patient. The distal end of the instrument in many instances has a fixed and invariable shape or contour and includes a pressurizable pressure tube providing a lumen for conveying the pressurized liquid, and a nozzle, through which the pressurized liquid exits to form a liquid jet. These instruments may include an evacuation tube providing an evacuation lumen, which receives some or all of the liquid from the jet, as well as excised tissue, and removes such materials for disposal. The evacuation tube may have a diameter considerably larger than the diameter of the pressure tube. In some of these instruments, the jet is emitted “proximally”, i.e., in a direction back towards the handle. In other configurations, the jet may be emitted “laterally”, i.e. in a direction substantially perpendicular to the longitudinal axis of the pressure tube in regions proximal to the distal end of the instrument, “distally”, or at some intermediate angle.

While currently available surgical liquid jet instruments represent, in some instances, significant improvements over many prior art surgical instruments for performing open and minimally invasive surgical procedures, there remains a need in the art to provide liquid jet surgical instruments which have certain improved capabilities, and which have the ability to be utilized in a wide variety of open and minimally invasive surgical procedures. The present invention provides, in many embodiments, such improved surgical liquid jet instruments, and further provides methods for their use in a variety of surgical procedures.

### SUMMARY OF THE INVENTION

Disclosed herein are a series of devices related to surgical procedures utilizing liquid jets for cutting, ablating, sculpting, trimming, etc., tissues and/or materials from the body of a patient. The invention includes, in one aspect, a series of devices comprising surgical liquid jet instruments for forming a liquid jet, in another aspect, methods for using the surgical liquid jet instruments, and, in yet another aspect, methods for forming certain components of the surgical liquid jet instruments.

In one aspect, the invention provides a surgical instrument having a distal end adapted to perform a surgical procedure on a patient and a proximal end. The instrument includes a pressure tube having sufficient burst strength to conduct a high pressure liquid towards the distal end of the instrument, where the pressure tube includes at least one nozzle providing a jet opening, the nozzle being shaped to form a liquid jet as the high pressure liquid flows therethrough. The instrument further includes an evacuation tube including a jet-receiving opening locatable opposite the jet opening, where the nozzle is aligned with the jet-receiving opening to receive the liquid jet when the instrument is in operation. At least the distal ends of both the pressure and evacuation tubes have a first configuration in a non-relaxed state and a second configuration in a more relaxed state. A straightener is constructed to selectively control the configuration of the distal ends of both the pressure and evacuation tubes to change one or more of the radius of curvature of the distal ends of both the pressure and evacuation tubes, the arc length of a curved portion of the distal ends of both the pressure and evacuation tubes, and the angular orientation with respect to a longitudinal axis of the proximal end of the surgical instrument of both a center line of the nozzle and a center line of the jet-receiving opening.

In yet another aspect, the invention provides a method comprising inserting at least a portion of a surgical liquid-jet instrument comprising a distal end adapted to perform a surgical procedure on a patient and a proximal end into a surgical site in the body of a patient. Relative motion between a straightener and a pressure tube and an evacuation tube of the surgical liquid-j et instrument is created where at least the distal ends of both the pressure and evacuation tubes undergo bending or straightening as the straightener and the pressure and evacuation tubes are moved relative to each other. One or more of the radius of curvature of the distal ends of both the pressure and evacuation tubes, the arc length of a curved portion of the distal ends of both the pressure and evacuation tubes, and the angular orientation of the center lines of both a nozzle in fluid communication with the pressure tube and a jet-receiving opening of the evacuation tube with respect to a longitudinal axis of the proximal end of the surgical instrument changes as the tubes bend or straighten. A liquid jet is created with the surgical liquid-jet instrument by flowing a liquid under high pressure through the nozzle in fluid communication with the pressure tube and the liquid jet is directed towards the jet-receiving opening of the evacuation tube of the surgical liquid-jet instrument to cut or ablate a selected tissue within the surgical site with the liquid jet.

In another aspect, the invention provides a method comprising inserting at least a portion of a surgical liquid-jet instrument comprising a distal end adapted to perform a surgical procedure on a patient and a proximal end into a surgical site in the body of a patient, where the distal end of the surgical liquid-jet instrument is in a first configuration as it is inserted into the surgical site. The distal end of the surgical liquid-jet instrument is deployed into a second configuration, where the distal end of the surgical liquid-jet instrument comprises a pressure tube and an evacuation tube which undergo bending or straightening as the instrument is deployed into the second configuration, and the shape of the distal end of the pressure and evacuation tubes is specifically adapted for the surgical site when in the deployed configuration. A liquid jet is created with the surgical liquid-jet instrument by flowing a liquid under high pressure through a nozzle in fluid communication with the pressure tube and the liquid jet is directed towards a jet-receiving opening of the evacuation tube of the surgical liquid-jet instrument to cut or ablate a selected tissue within the surgical site with the liquid jet.

In yet another embodiment, the invention provides a method of manufacturing a surgical liquid-jet instrument comprising a pressure tube and an evacuation tube. The method comprises forming a bend in the distal end of a pressure tube of the surgical liquid-jet instrument, where the pressure tube has sufficient burst strength to conduct a high pressure liquid towards a distal end of the instrument, the pressure tube including at least one nozzle providing a jet-opening, where the nozzle is shaped to form a liquid jet as a liquid at high pressure flows therethrough. A bend is also formed in the distal end of an evacuation tube of the surgical liquid-jet instrument, where the evacuation tube includes a jet-receiving opening having a cross-sectional area located opposite the jet-opening. A straightener is slidably connected to at least the distal ends of both the pressure and evacuation tubes, where the straightener is constructed to selectively control the configuration of the distal ends of both the pressure and evacuation tubes to change one or more of the radius of curvature of the distal ends of both the pressure and evacuation tubes, the arc length of a curved portion of the distal ends of both the pressure and evacuation tubes, and the angular orientation with respect to a longitudinal axis of the proximal end of the surgical instrument of both a center line of the nozzle and a center line of the jet-receiving opening.

### BRIEF DESCRIPTION OF THE DRAWINGS

The accompanying drawings are schematic and are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In the drawings:

FIG. 1 is a schematic illustration of a surgical liquid jet system;

FIG. 2a is a partially-cutaway schematic illustration of a surgical liquid jet instrument;

FIG. 2b is a detailed schematic illustration of a portion of the distal end of the surgical liquid jet instrument in FIG. 2a in a deployed configuration;

FIG. 2c is a detailed schematic illustration of a portion of the distal end of the surgical liquid jet instrument in FIG. 2a in a different deployed configuration;

FIG. 2d is a detailed schematic illustration of a portion of the distal end of another embodiment of a surgical liquid jet instrument;

FIG. 3a is a partially-cutaway schematic illustration of a portion of the distal end of a surgical liquid jet instrument;

FIG. 3b is a schematic cross-sectional illustration of the surgical liquid jet instrument taken along line 3b-3b in FIG. 3a;

FIG. 4 is a schematic cross-sectional illustration of a portion of the distal end of one embodiment of a surgical liquid jet instrument;

FIG. 5a is a schematic illustration of a portion of the distal end of another embodiment of a surgical liquid jet instrument;

FIG. 5b is a schematic illustration of a portion of the distal end of yet another embodiment of a surgical liquid jet instrument in a first configuration;

FIG. 5c is a schematic illustration of the portion of the distal end of the embodiment shown in FIG. 5b in a second configuration;

FIG. 6 is a partially-cutaway schematic illustration of one embodiment of an advancing mechanism to control the relative movement of the straightener relative to the pressure tube and evacuation tube;

FIG. 7 is a schematic illustration of a surgical liquid jet system according to another embodiment; and

FIG. 8 is a schematic cross-sectional illustration of a surgical liquid jet instrument inserted into the spine of a patient.

### DETAILED DESCRIPTION

The present invention provides a variety of liquid jet instruments useful in a variety of applications, many of which instruments are especially well suited for a variety of surgical procedures. Certain embodiments of the liquid jet instruments provided by the invention can be configured in a variety of different ways for use in various surgical operating fields. Certain surgical instruments, according to the invention, are configured as surgical handpieces having a proximal end with a grasping region, or handle, shaped and configured to be comfortably held by the hand of an operator. The instruments may also have a distal end that includes at least one nozzle for forming a liquid jet. The distal end of certain embodiments of the inventive surgical instruments can be used to perform a surgical procedure on a patient. Although the liquid jet instruments described herein are shown as having a handpiece configuration, it should be understood that the invention is not strictly limited to surgical handpieces, and that the invention may also be practiced utilizing liquid jet instruments having a variety of configurations and purposes. Certain embodiments of the liquid jet instruments provided by the invention can be used in a wide variety of surgical applications to utilize a high pressure liquid stream to cut, drill, bore, perforate, strip, delaminate, liquefy, ablate, shape, or form various tissues, organs, etc. of the body of a patient.

At the outset, it should be noted that a detailed treatment and discussion of a wide variety of design parameters, configurations, materials of construction, and other aspects of the design, fabrication, and construction of liquid jet surgical instruments are provided in commonly owned U.S. Pat. Nos. 5,944,686; 6,375,635; 6,511,493; 6,451,017; 7,122,017; and 6,960,182; in U.S. Patent Application Publication Numbers 2003/0125660 A1, US2002-0176788 A1, US2004-0228736 A1, 2004/0243157 A1, US2006-0264808 A1, and US2006-0229550, each of which is incorporated herein by reference. The reader is referred to these issued patents and patent publications for detailed description of and guidance as to the construction and design of certain embodiments of the liquid jet components of the instruments described herein. For example, U.S. Pat. No. 6,375,635 describes in detail design considerations related to the configuration and sizing of the nozzle, evacuation lumen, liquid jet length and dispersion, materials of construction, liquid pressures for operation, etc. for liquid jets configured to directly contact, cut and/or fragment and/or disaggregate tissue and facilitate removal of tissue through an evacuation lumen. Accordingly, while certain specific design parameters are called out and discussed in more detail below, others that may not specifically mentioned or discussed are discussed in detail in one or more of the above-referenced U.S. Patents or Patent Publications. Such parameters, configurations and design considerations disclosed in these references can be, in many cases, applicable to and useful for practicing many aspects of the current invention. Moreover, while certain features provided according to the present invention are illustrated for the purpose of exemplification in context of a few illustrated instrument designs and configurations, it should be understood that such features may be useful, in many instances, in the context of other instrument designs; such as, for example, instrument designs disclosed in the commonly owned patents and applications listed directly above.

Certain embodiments of the liquid jet surgical instruments provided by the invention include a pressure tube, having a terminal end defining, forming or circumscribing at least one nozzle providing a liquid jet opening, and having a proximal end that is connectable to a source of liquid under high pressure, supplied, for example, by a high pressure pump or liquid dispenser. The liquid jet nozzle is shaped to form a liquid jet as a liquid under high pressure flows through the nozzle, as described below. The liquid jet, in certain embodiments, can be used to cut, ablate, sculpt, trim, form, debride, etc., various tissues of a patient in surgical procedures. In certain embodiments, the liquid pressure supplied to the instrument by the pump or dispenser is variably controllable by an operator of the instrument so that the cutting or ablating power of the liquid jet is adjustable by the operator. This adjustability of the pressure can allow an operator to create a liquid jet with the instrument that can differentiate between different types of tissue within a surgical operating field. For example, a lower pressure can be utilized for cutting or ablating a soft tissue such as fat or the nucleus pulposus of an intervertebral disc from a surface of a harder tissue, such as muscle, bone, cartilage, or the annulus fibrosus of an intervertebral disc, where the liquid jet has sufficient strength to cut or ablate the soft tissue without damaging the underlying, surrounding, adjacent, and/or interdigitated harder tissue. A higher pressure can then be selected that is sufficient to form a liquid jet capable of cutting or ablating hard tissue, such as muscle or bone. In this way, a liquid jet surgical instrument provided by certain embodiments of the invention can provide highly selective and controllable tissue cutting in various surgical procedures, such as, for example, surgical procedures on the spine.

Various embodiments of the invention are directed to liquids jet surgical instruments in which the distal end has a selectively controllable shape. The surgical instrument may include a pressure tube and an evacuation tube where the distal ends of these tubes may have a first configuration in a non-relaxed state and a second configuration in a more relaxed state. An operator may change the shape of the distal ends of the pressure tube and evacuation tubes between the first configuration and the second configuration based upon the needs and spatial constraints of the particular surgical procedure or operating space. In some embodiments, an operator may change the shape of the distal end of the instrument during the surgical procedure based upon the size and shape of the surgical site.

The surgical instrument in certain embodiments includes a straightener to selectively control the configuration of the distal ends of both the pressure and evacuation tubes. As discussed in greater detail below, in certain embodiments, the straightener is constructed to change the radius of curvature and/or arc length of the distal ends of the tubes. In the same or other embodiments, the straightener is constructed to change the angular orientation of the distal ends of the tubes.

The “longitudinal axis” of the jet nozzle, as will be described in more detail below, is defined by the axial center line of the nozzle region of the pressure tube, which is typically at the terminal tip of the pressure tube. The “longitudinal axis” of the jet-receiving opening is defined by the axial center line of the jet-receiving opening of the evacuation tube. The “longitudinal axis” of the evacuation lumen refers to an axis defining the geometric center of the evacuation lumen in a region that is proximal to the jet-receiving opening. In typical embodiments, this region of the evacuation lumen will have a longitudinal axis that is essentially parallel to the longitudinal axis of the elongated body of the instrument, which is held and controlled by the hand of the operator. As used herein in the context of describing geometric relationships between longitudinal axes of various components, the term “co-linear” refers to components whose longitudinal axes are superimposed on essentially the same line in space. The term “parallel” when used in the same context herein refers to longitudinal axes that are not co-linear, but that are oriented in an essentially identical direction in space.

The inventive surgical liquid jet instruments will now be described in more complete detail in the context of several specific embodiments illustrated in the appended figures. It is to be understood that the embodiments described are for illustrative purposes only and that the novel features of the invention, as described in the appended claims, can be practiced in other ways or utilized for instruments having other configurations, as apparent to those of ordinary skill in the art.

Aspects of the present invention are directed to a surgical instrument where at least the distal ends of both the pressure tube 110 and evacuation tube 112 can be controllably adjusted to provide a distal end with more than one shape and/or other configuration. The distal ends of the pressure and evacuation tubes may have one configuration/shape in a relatively relaxed state and another different configuration/shape in a relatively more non-relaxed state. A “non-relaxed state,” “relatively non-relaxed state,” or “less relaxed state” as used herein may be defined as the condition of the tubes when a force from another component acts on the tubes in a way which can substantially alter the shape the distal ends of the tubes would have but for the application of the force. A “relaxed state,” “relatively relaxed state,” or “more relaxed state” as used herein may thus be defined as the condition of the tubes without the above-mentioned force from another component acting on the tubes or with a lower level of such force acting on the tubes and/or with the same or a lower level of such force applied but acting on a smaller portion of the overall length of the tubes than when the tubes are in the non-relaxed state, relatively non-relaxed state, or “less relaxed state.”. In one embodiment, the distal ends of both the pressure and evacuation tubes are substantially straight in a relatively non-relaxed state.

As discussed in greater detail below, in some embodiments, the distal ends of both the pressure and evacuation tubes may be configured and shaped such that in a relatively relaxed state the distal ends of the tubes have a preformed bent configuration. The bent configuration may be defined by the degree of bending. The “degree of bending” of the distal ends of the pressure and evacuation tubes is the amount to which the tubes deviate from a straight form. The bending may result in a distal end having a curved configuration, a distal end having an angled configuration characterized by at least two substantially straight interconnected or materially continuous segments angled relative to each other, or a distal end having a combination of both a curved and angular configuration.

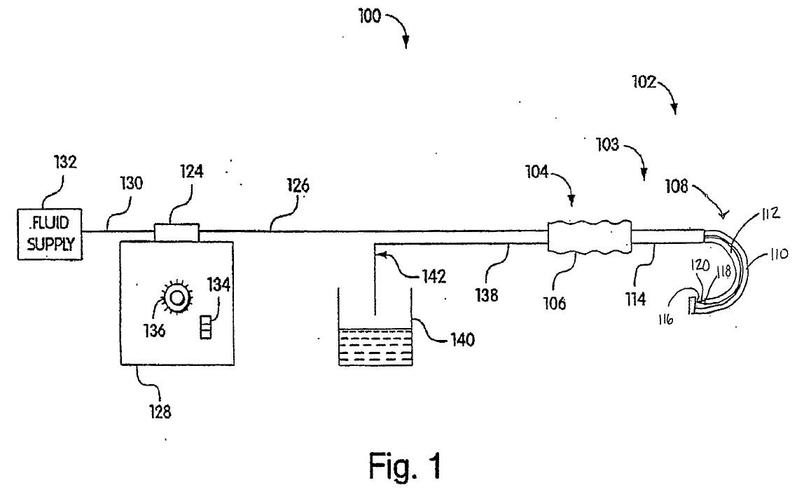

FIG. 1 shows one embodiment of a liquid jet surgical system 100 utilizing a liquid jet surgical instrument 102, according to-an embodiment of the invention. The surgical instrument 102 is configured as a surgical handpiece having a proximal end 103 including a body 104 having a grasping region 106 configured for placement in the hand of an operator of the instrument. The surgical instrument 102 has a distal end 108 including a pressure tube 110 forming a pressure lumen and an evacuation tube 112 forming an evacuation lumen. “Distal end” when used herein in the context of a region of a surgical instrument refers to the portion of the surgical instrument that is adapted to perform a surgical procedure on a patient, and which is inserted into a surgical site during operation of the instrument. The distal end 108 of the instrument 102 may, in some embodiments, comprise only the distal ends of pressure tube 110 and evacuation tube 112, or in other embodiments, may include components proximal to the distal ends of the pressure tube 110 and the evacuation tube 112 that are also inserted into a surgical operating space of the patient during use of the instrument. In the illustrated embodiment, surgical instrument 102 further includes a straightener, whose function is explained in more detail below, in the form of sheath 114, which at least partially surrounds pressure tube 110 and evacuation tube 112 and supplies support for the tubes to assist in maintaining and/or establishing a desired geometric configuration between the pressure tube and the evacuation tube, when the instrument 102 is in operation. The pressure lumen formed by tube 110 further includes at the terminal tip at its distal end a nozzle 116, which forms a liquid jet as a high pressure liquid supplied by pressure tube 110 streams therethrough. The evacuation lumen formed by tube 112 includes a jet-receiving opening 118 located at the terminal tip at its distal end and positioned, when the instrument 102 is in operation, opposite the jet nozzle 116 at a predetermined distance therefrom in order to receive the liquid jet 120.

The pressure tube 110 and evacuation tube 112 may be constructed from a variety of materials which are discussed in greater detail below. Regardless of the specific material from which the pressure tube is constructed, the pressure tube should have sufficient burst strength to enable it to conduct a liquid to nozzle 116 at the pressures contemplated for operation to form liquid jet 120. The burst strength of the pressure tube should be selected to meet or exceed the highest contemplated pressure of the liquid supplied for use in the specific surgical procedure to be performed. Typically, surgical instrument 102 will operate at liquid pressure between about 500 psig and about 50,000 psig, depending on the intended material to be cut and/or ablated.

Pressure tube 110 is in fluid communication with high pressure pump 124 via high pressure liquid supply conduit 126. High pressure liquid supply conduit 126 also should have a burst strength capable of withstanding the highest liquid pressures contemplated for using the instrument 102 for a particular surgical application. In some embodiments, high pressure liquid supply conduit 126 comprises a burst-resistant stainless steel hypotube constructed to withstand at least 50,000 psig.

In fluid communication with high pressure liquid supply conduit 126 is a high pressure pump 124, which can be any suitable pump capable of supplying the liquid pressures required for performing the desired surgical procedure. Those of ordinary skill in the art will readily appreciate that many types of high pressure pumps may be utilized for the present purpose, including, but not limited to, piston pumps and diaphragm pumps. In certain embodiments, high pressure pump 124 comprises a disposable piston or diaphragm pump, which is coupled to a reusable pump drive console 128. High pressure pump 124 has an inlet that is in fluid communication with a low pressure liquid supply line 130, which receives liquid from liquid supply reservoir 132. Pump drive console 128 may include an electric motor that can be utilized to provide a driving force to high pressure pump 124 for supplying a high pressure liquid in liquid supply conduit 126.

While a variety of known pump consoles may be utilized in the context of the present invention, certain pump drive consoles include a constant speed electric motor that can be turned on and off by means of an operator-controlled switch 134. In certain embodiments, operator-controlled switch 134 comprises a foot pedal or a button or trigger located on grasping region 106 of the surgical instrument 102 that may be easily accessed by the operator of the instrument. In some embodiments, the pressure/flow rate may be controlled by the operator via an adjustable pressure/flow rate control component 136, that can control the motor speed of the pump drive console and/or the displacement of the high pressure pump. While in FIG. 1, pressure/flow rate control component 136 is illustrated as a knob on pump drive console 128, in certain embodiments, such component would comprise a foot pedal, or trigger/button located on grasping region 106, as previously discussed for on/off control of the pump drive console 128. In yet other embodiments, pump drive console 128 and high pressure pump 124 may be replaced by a high pressure liquid dispenser or other means to deliver a high pressure liquid, as apparent to those of ordinary skill in the art. In certain embodiments, a pumping system such as one of those described in commonly-owned U.S. Patent Application Publication Nos. 2002/0176788, or 2004/0228736, both incorporated herein by reference, could be used.

The liquid utilized for forming the liquid cutting jet can be any fluid that can be maintained in a liquid state at the pressures and temperatures contemplated for performing the surgical procedures. For applications in which the instruments are used to perform surgical procedures in a live patient, the liquid utilized should also be physiologically compatible. In typical embodiments, the liquid supplied will be a sterile surgical saline solution, or sterile water and liquid supply reservoir 132 can comprise a sterile container, such as an intravenous (IV) bag containing such fluid. In some embodiments, in order to improve the cutting or ablating character of the liquid jet, the liquid may contain solid abrasives, or the liquid may comprise a liquefied gas, for example carbon dioxide, which forms solid particulate material upon being admitted from nozzle 116 to for the liquid jet 120. In other embodiments, the liquid supplied to surgical instrument 102 may include medicaments, such as antiseptics, antibiotics, antiviral components, anesthetics, drugs, chemotherapy agents, etc., that are useful in the context of a specific surgical procedure. In other embodiments, the fluid may include a dye to improve visualization of the liquid jet when the instrument is in operation.

Evacuation tube 112 is connectable at its proximal end to an evacuation conduit 138, which can be used to transport evacuated material and debris to a drainage reservoir 140. The liquid contained in evacuation conduit 138 is typically under relatively low pressure and, accordingly, evacuation conduit 138 may be constructed, in certain embodiments, of a low cost flexible material, for example, polymeric tubing, such as polyvinyl chloride (PVC), silicone, polyethylene, rubber, etc. tubing. In certain embodiments, evacuation conduit 138 should have a minimum internal cross-sectional area that equals or exceeds the maximum internal cross-sectional area of the evacuation lumen.

In certain embodiments, the evacuating force created by the liquid jet being directed into the evacuation lumen is sufficient to evacuate material from the operating site to a drainage reservoir located at the proximal end of the evacuation tube or an evacuation conduit connected to the proximal end of the evacuation tube. In such embodiments, the liquid jet and the evacuation tube together can act as an eductor pump, which utilizes the momentum and kinetic energy of the moving fluid of the liquid jet to create an evacuating force capable of driving the liquid, ablated material, and debris through the evacuation lumen and away from the surgical site. The reader is directed to commonly owned U.S. Pat. No. 6,375,635 and U.S. Published Patent Application No. 2004/0243157 for a more detailed discussion. In the illustrated embodiment, surgical instrument 102 is constructed such that the evacuation lumen is capable of evacuating liquid jet 120 and ablated material and debris from the jet-receiving opening 118 to the proximal end of the evacuation lumen and through evacuation conduit 138 into drainage reservoir 140, without the need for an external source of suction. In such embodiments, evacuation conduit 138 may include a vacuum breaker 142 or a proximal end that is not couplable to an external source of suction, so that it is not possible for an operator to inadvertently couple evacuation conduit 138 to an external source of suction when the instrument is in operation. In other embodiments, an external source of suction, for example a vacuum pump or aspirator, can be provided in fluid communication with a proximal end of an evacuation lumen of an evacuation tube of the instrument in order to provide the suction driving force required for evacuating material from the surgical field via a jet-receiving opening of the evacuation tube.

In certain embodiments, the fluid supply path of liquid jet surgical system 100 is disposable, and sterilizable, for example by chemical methods such as exposure to ethylene oxide, or by gamma or beta irradiation, as apparent to those of ordinary skill in the art. In certain embodiments, the fluid path is supplied pre-sterilized to the user for a single use only. Those of ordinary skill in the art understand what is meant by “disposable” and “for a single use only.”

The present invention provides, in certain embodiments, surgical liquid jet instruments which are specifically designed and constructed for use in a particular surgical environment. Specifically, in some embodiments, the present invention provides surgical liquid jet instrument designs that are tailored to provide highly desirable performance characteristics in surgical operating environments where the liquid jet is submerged in a liquid environment when the instrument is in operation, and, in other embodiments, the present invention provides surgical liquid jet instrument designs that are tailored to provide highly desirable performance characteristics in surgical operating environments where the liquid jet is surrounded by a gaseous environment when the instrument is in operation. The reader is referred to U.S. Pat. No. 6,375,635 and US. Published Application No. 2006/0229550 A1 for detailed description of and guidance as to the construction and design of certain embodiments of the liquid jet components of the instruments based upon the nature of the surgical environment.

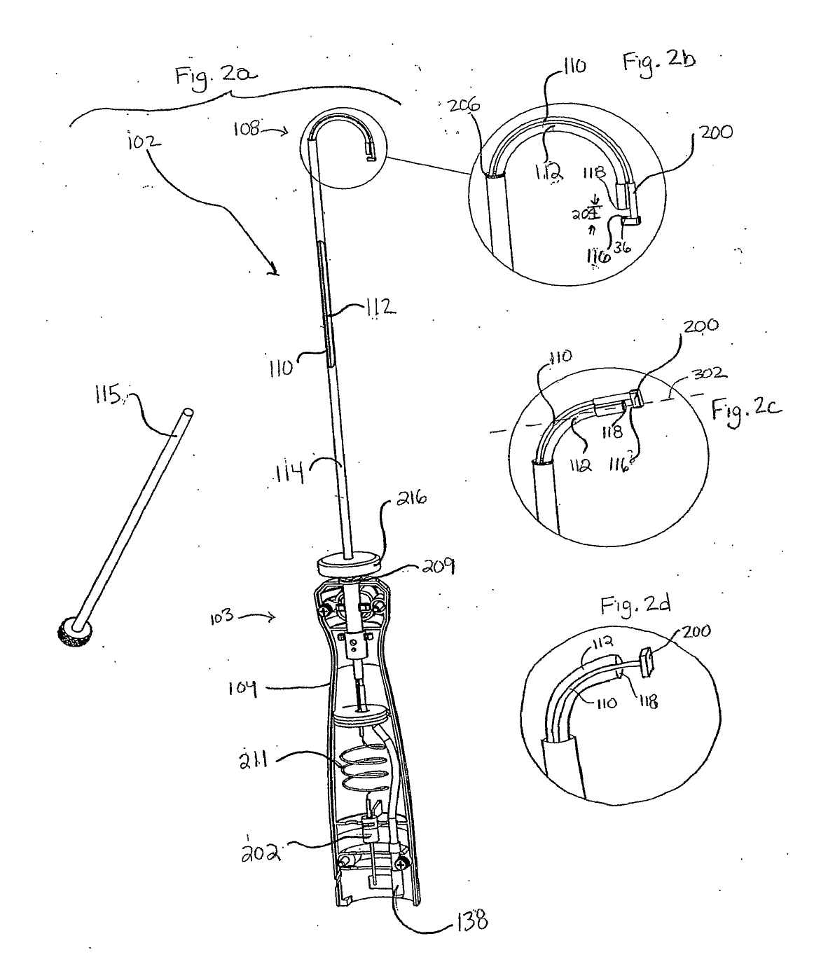

FIG. 2a-2b illustrate one configuration of liquid jet surgical instrument 102, according to an embodiment of the invention. The instrument 102 includes a pressure tube 110 and an evacuation tube 112. As shown in the partial cutaway view in FIG. 2a, the instrument is configured as a handpiece with a body 104 that may be provided at the proximal end 103 of the instrument 102. The handpiece has a body 104 and the proximal end of the pressure tube 110 and evacuation tube 112 pass through the body of the handpiece instrument 102. In some embodiments, the handpiece body 104 may help an operator use and control the instrument. In some embodiments the body 104 may include various controls, such as on/off switches. In the particular embodiment illustrated in FIG. 2a, a portion of the proximal end 103 of the evacuation tube 112 is rigidly coupled to the handpiece body 104. However, in other embodiments, the instrument 102 may not include be configured as a handpiece and may lack a body (e.g. the instrument may be configured as an elongated catheter).

At the distal end 108, the pressure tube 110 includes at least one nozzle 116 providing a jet-opening, and the evacuation tube 112 includes a jet-receiving opening 118 locatable opposite the jet opening. In the particular embodiment illustrated in FIGS. 2a-2b, the nozzle 116 is formed in a manifold 200 which is coupled to the pressure tube 110. The manifold 200 is discussed in greater detail below, but as shown, in some embodiments, the manifold couples the distal end of the pressure tube 110 to the distal end of the evacuation tube 112 to prevent relative motion therebetween during straightening and bending of the distal end 108 of the tubes.

As shown, the nozzle 116 is aligned with the jet-receiving opening 118 to receive a liquid jet when the instrument 102 is in operation. A filter 202 may be provided at the proximal end of the pressure tube 110 to prevent contaminants from plugging the nozzle 116. As discussed previously, the pressure of the high pressure liquid supplied to nozzle 116 for forming the liquid jet depends on the particular design of nozzle 116 and the hardness/toughness of tissue or material to be cut or ablated. In certain embodiments, the liquid at high pressure is supplied to the jet opening at a pressure of at least 500 psig, in other embodiments at a pressure of at least about 1,000 psig, 2,000 psig, 3,000 psig, or 5,000 psig, and still other embodiments at a pressure of at least about 10,000 psig, or 15,000 psig, or within a range of about 10,000 psig to 20,000 psig, and still other embodiments at a pressure of at least 20,000 psig, and in yet still other embodiments at a pressure of at least about 30,000 psig, or 50,000 psig. It is contemplated that the present invention may incorporate various types of nozzle-forming technology, such as that described in commonly-owned U.S. Pat. No. 6,375,635 and U.S. Published Application No. 2006/0264808 which are both herein incorporated by reference in their entireties. The jet opening can have a circular cross-sectional area, but may, in other embodiments, have other cross-sectional shapes, such as rectangular, oval, slit-like, etc., for forming jets having different shapes for specific desired purposes.

Certain embodiments of the inventive liquid jet surgical instruments may include distal ends that are designed and configured to prevent or reduce plugging of the evacuation lumen, blow-by of the liquid jet, or back spray or misting of the liquid jet when the instrument is in operation. “Blow-by” of the liquid jet, as used herein, refers to a portion of the liquid jet, or a high velocity fluid entrained by the liquid jet having a cross-sectional area, at the plane of the jet-receiving opening, that is larger than the cross-sectional area of the jet-receiving opening so that at least a portion of the liquid jet or high velocity fluid misses or “blows by” the jet-receiving opening. Blow-by is generally undesirable because it can lead to unintended tissue damage and poor evacuation efficiency. “Back spray” as used herein refers to a liquid jet, or high velocity fluid entrained by the liquid jet, entering the jet-receiving opening in the evacuation tube and subsequently reflecting or flowing back into the surgical field from the jet-receiving opening. Such back spray is undesirable in operation due to the potential of contamination of the surgical operating field and/or aerosolization of infective material, in addition, back spray typically indicates a poor efficiency level of the evacuation of material by the instrument via eductor pump action. As described in more detail in U.S. Pat. No. 6,375,635, the surgical instrument may be configured in various ways to in certain embodiments, substantially reduce, and in certain embodiments essentially eliminate, performance problems associated with blow-by and back spray when the instruments are in operation.

At least the distal ends of both the pressure tube 110 and evacuation tube 112 can advantageously be made of a material which is able to withstand repeated movement between relatively relaxed and relatively non-relaxed states. In one embodiment, at least the distal ends of the pressure and evacuation tubes 110, 112 are made of a metal, in certain instances an elastic, very elastic, or superelastic metal, as those terms would be understood by those skilled in the art. In one embodiment, at least the distal ends of the pressure and evacuation tubes are made of a material such as stainless steel, tungsten, nickel-titanium, Monel® which is a stainless steel alloy composed primarily of nickel and copper manufactured by Special Metals Corporation, Inconel® which is a nickel-based alloy manufactured by Special Metals Corporation, Hastelloy® which is another metal alloy manufactured by Haynes International, Inc., Elgiloy® which is another metal alloy manufactured by Elgiloy Specialty Metals Corporation, MP35N® another metal alloy manufactured by SPS Technologies, Inc., or 35NLT® yet another metal alloy manufactured by Fort Wayne Metals. In another embodiment, at least the distal end of the pressure and evacuation tubes is made of a superelastic material, such as nickel-titanium alloys. In one embodiment, a material known as NITINOL may be used. NITINOL is an acronym for Nickel Titanium Naval Ordnance Laboratory, and refers to a family of intermetallic materials which contain nearly an equal mixture of nickel and titanium. NITINOL is able to recover strains on the order of magnitude of 8% which makes this material superelastic. In yet another embodiment, non-metal elastic materials, such as rigid elastic polymeric materials, may be used to form at least the distal ends of the pressure and evacuation tubes.

In one embodiment, the bent configuration of the distal ends of the pressure and evacuation tubes may be formed by a heat treatment process. The particular method of heat treatment utilized may vary based upon the particular material comprising the tubes, but in one embodiment, wherein the desired bend is formed into a pressure tube 110 made of NITINOL tubing with a wall thickness of approximately 0.006 inches (0.15 mm), heat treatment at a temperature of approximately 750° F.-770° F. (405° C.-415° C.) for about 10-12 minutes is performed with the tube immobilized in a configuration having the desired degree of bending. Thereafter, and with the desired degree of bending maintained, the tube 110 is quenched in a cooling fluid, such as water, at a temperature of approximately 55° F. For a thicker walled tubing, such as a NITINOL pressure tube 110 having a wall thickness of approximately 0.020 inches (0.5 mm) and an outer diameter of approximately 0.080 inches (2 mm), the bend may be formed at a temperature of approximately 975° F. (525° C.) for about 12 minutes, followed by quenching in a cooling fluid, such as water.

In one embodiment, the proximal end of one or both of the pressure tube 110 and evacuation tube 112 may be formed from a different material than the distal end of the pressure tube 110 or evacuation tube 112. For example, in the embodiment illustrated in FIG. 2a, evacuation conduit 138 connects to the proximal end of the evacuation tube 112 and leads the waste fluid to a drain or tissue disposal reservoir. The shape of the distal end of the pressure tube and evacuation tube is configured to be controllably changed by an operator of the instrument, whereas the shape of the proximal end of these tubes may be configured to remain substantially the same. In some embodiments, the proximal end of these tubes may be formed from polymeric tubing, such as polyvinyl chloride (PVC), silicone, polyethylene, rubber, etc., so long as the material chosen has the capability of withstanding contemplated operating pressures and stresses.

In some embodiments, the surgical instrument includes a straightener to enable the configuration of the distal ends of both the pressure and evacuation tubes to be selectively controlled. The straightener may be constructed to enable a user of the instrument to selectively alter the configuration and/or shape of the distal ends of the pressure and evacuation tubes by, for example, deploying the tubes from a relatively non-relaxed state to a more relaxed state or visa versa. The straightener may, for example, effect such deployment by altering the degree of bending and/or the radius of curvature and/or arc length of the distal ends of the tubes 110, 112.

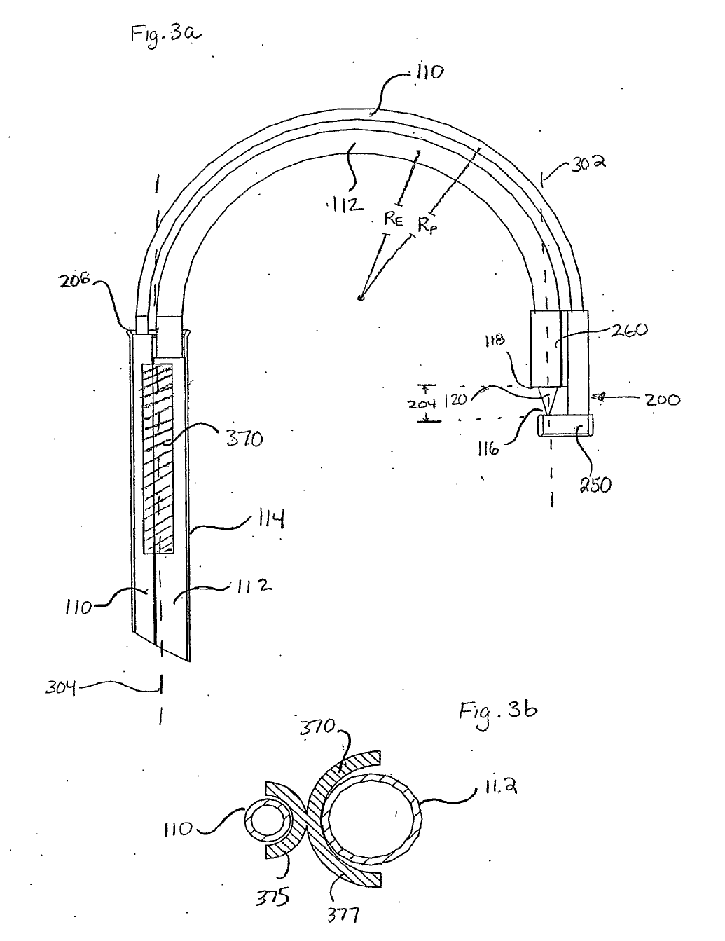

In certain embodiments, the straightener is constructed to change the radius of curvature of the distal ends of both the pressure and evacuation tubes. For example, in a first configuration, the radius of curvature of the distal ends of the pressure and evacuation tubes may be essentially infinite if the distal ends are substantially straight. In a second configuration, the radius of curvature of the distal ends of the pressure and evacuation tubes may be within approximately 9-20 mm and may for example be approximately 10 mm as the distal ends curve, where radius of curvature may be defined as the quantity that describes a radius of a circle whose circumference would match the shape of the distal end of the tubes 110, 112. The radius of curvature R_{P} and R_{E} for both the pressure tube and the evacuation tube is illustrated for one configuration illustrated in FIG. 3a.

The straightener may also be constructed to change the angular orientation of both a centerline 302 of the nozzle and a centerline 302 of the jet-receiving opening with respect to a longitudinal axis 304 of the proximal end of the surgical instrument. The angular orientation of the centerlines of the nozzle and jet-receiving opening with respect to a longitudinal axis of the proximal end of the surgical instrument is a measure of the angular movement of the distal end of the pressure and evacuation tubes with respect to the proximal end of the instrument. The angular orientation of both the nozzle 116 and the jet-receiving opening 118 changes as the distal ends of the tubes bend more with respect to the proximal end of the instrument.

It should be understood that in some embodiments, the straightener may be constructed to change each of the radius of curvature of the distal ends of both the pressure and evacuation tubes, the arc length of a curved portion of the distal ends of both the pressure and evacuation tubes, and the angular orientation with respect to a longitudinal axis of the proximal end of the surgical instrument of both a center line of the nozzle and a center line of the jet-receiving opening. However, in other embodiments, the straightener may be constructed to change only one of the radius of curvature, the arc length, and the angular orientation For example, in an embodiment in which the distal end of the pressure and evacuation tubes are bent in such a way that they are angularly deflected upon deployment without becoming curved, the straightener would change the angular orientation of the distal end of the tubes with respect to the longitudinal axis of the proximal end of the instrument, but the radius of curvature of the distal end of the tubes may remain essentially infinite and there would be no change in arc length, since the tubes are not curved.

It should be appreciated that in some embodiments, once the distal ends of the tubes are deployed from the straightener, the distal ends of the tubes have a radius of curvature that may no longer change as the distal ends of the tubes are further deployed from the straightener. Further deployment of the distal ends of the tubes, however, may increase the arc length of the curved portion of the distal ends of the tubes, which may change the angular orientation of the centerlines of the nozzle and the jet-receiving opening with respect to the longitudinal axis of the proximal end of the instrument.

The straightener may be configured differently according to different embodiments of the present invention, as would be appreciated by the skilled artisan, and only a limited number of exemplary embodiments are illustrated and discussed in detail below in the interests of brevity. In the particular embodiment illustrated in FIGS. 2a-2c, a tubular sheath 114 acts as a straightener. This sheath 114 surrounds at least a portion of the pressure tube 110 and evacuation tube 112. At least one of the sheath 114 and the pressure and evacuation tubes 110, 112 are slidable with respect to the other to effect a change in the shape of the distal end of both the pressure tube 110 and evacuation tube 112 (e.g. the sheath 114 may be fixed in position and the tubes may slide within the sheath, the tubes may be fixed and the sheath may slide with respect to the tubes, or both the sheath and the tubes may be slidable with respect to each other. In one embodiment, the tubular sheath 114 has a substantially circular cross-section. In another embodiment, the sheath 114 may have a substantially ovoid shape which may assist in the alignment of the pressure tube 110 with respect to the evacuation tube 112.

In the embodiment illustrated in FIG. 2a, the sheath 114 is positioned adjacent to and permanently connected to the handpiece body 104. In another embodiment, sheath 114 may not form a part of the assembled instrument 102, but instead, the distal portion of the instrument may be reversibly inserted into a tubular sheath that is a separate element, such as a cannula 115, which acts as a straightener. Such a configuration is particularly applicable for instruments configured as laproscopic instruments and catheters.

In one embodiment, the sheath 114 is slidable with respect to the pressure and evacuation tubes 110, 112. As the sheath 114 is moved proximally relative to the pressure and evacuation tubes 110, 112 the degree of bending may increase and/or the radius of curvature of the distal ends of the tubes may decrease, at least until a final radius of curvature indicative of a more-relaxed or completely relaxed configuration is achieved. As the sheath 114 is moved distally relative to the pressure and evacuation tubes 110, 112 the distal ends of the tubes may straighten.

In yet another embodiment, the pressure and evacuation tubes 110, 112 are slidable with respect to the sheath 114. In this embodiment, the degree of bending may increase and/or the radius of curvature of the distal ends of the tubes may decrease as the tubes are moved distally relative to the sheath 114, whereas the tubes may straighten as the tubes are moved proximally, retracting back into the sheath 114.

To facilitate the relative movement between the sheath 114 and the pressure and evacuation tubes 110, 112, in some embodiments, the tip 206 of the sheath 114 may flare outwardly. Furthermore, the inside surfaces of the sheath 114 and/or the outside surfaces of the pressure and/or evacuation tubes 110, 112 may have a lubricant coating to facilitate the relative sliding movement.

FIG. 2c illustrates the distal end 108 of the instrument 102 shown in FIGS. 2a-2b in another different configuration. The centerline 302 of the nozzle 16 and the centerline 302 of the jet-receiving opening 118 at the distal end of the instrument shown in FIG. 2c have a different angular orientation with respect to the proximal end of the instrument in comparison to the distal end in the position illustrated in FIG. 2b. Furthermore, the radius of curvature in the distal end of the instrument as shown in FIG. 2c is greater than the radius of curvature of the distal end of the instrument as shown in FIG. 2b. The configuration of the instrument 102 as shown in FIG. 2c may be more suitable for certain surgical procedures, whereas the configuration illustrated in FIGS. 2a-2b may be more suitable for other surgical procedures. By controlling relative movement between the tubular sheath 114 and the pressure and evacuation tubes 110, 112 an operator may change the configuration of the distal end of the instrument based upon the needs of a particular surgical procedure or surgical operating space. Furthermore, the operator may, in certain embodiments, change the shape of the distal end from one configuration to another configuration while the distal end of the instrument 102 is within a surgical site in the body of a patient.

In another configuration the distal end of the pressure and evacuation tubes 110, 112 of the instrument 102 shown in FIGS. 2a-2c are fully retracted into the sheath 114 (not illustrated). In this configuration, the distal end of the pressure and evacuation tubes 110, 112 may be substantially straight such that the radius of curvature is essentially infinite and the angular orientation of the centerline of both the nozzle and the jet-receiving opening is substantially the same as the longitudinal axis of the proximal end of the instrument. In this straight relatively non-relaxed configuration, the longitudinal axis of the proximal end of the instrument is substantially collinear with or parallel to the centerline 302 of the nozzle and jet-receiving opening.

Turning to FIG. 3a, a detailed schematic view of a distal end of a surgical instrument with a manifold 200 coupled to the pressure tube 110 is illustrated. In this embodiment, the nozzle 116 is formed in the manifold 200. A lumen (not shown) is also formed within the manifold 200 such that when the manifold 200 is coupled in a leak-tight fashion, for example via welding, brazing, press fitting, gluing, or otherwise to the pressure tube 110, the pressure lumen of the pressure tube is in fluid communication with the nozzle 116. The nozzle 116 is located opposite the jet-receiving opening 118 such that the jet-receiving opening 118 receives the liquid jet 120. Although the manifold illustrated in FIG. 3a is coupled to the pressure tube 110, it should be appreciated that in other embodiments the manifold 200 may be formed integrally with the pressure tube 110. Furthermore, it is also contemplated that in some embodiments, a separate manifold 200 is not utilized and instead the nozzle 116 may be formed in the distal tip of the pressure tube 110 itself.

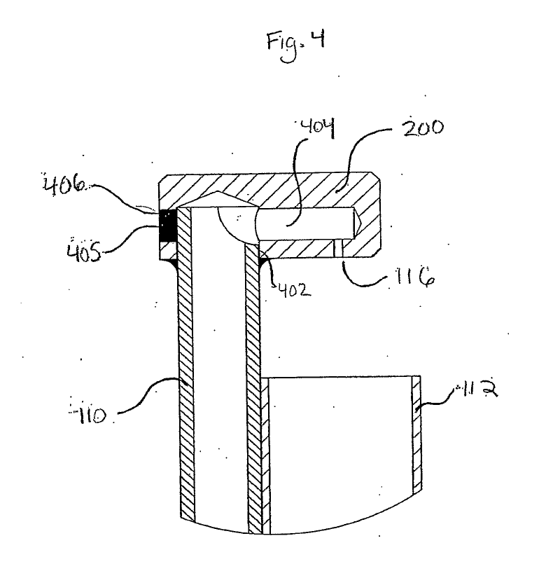

In the embodiment illustrated in FIG. 3a, the manifold 200 includes a sleeve 260 which couples the distal tip of the pressure tube 110 to the distal tip of the evacuation tube 112 to prevent relative motion therebetween as the shape of the distal end 108 of the instrument is deployed from a first configuration to a second configuration. Coupling the distal tip of the pressure tube 110 with the distal tip of the evacuation tube 112 can facilitate keeping the nozzle 116 aligned with the jet-receiving opening 118. In the illustrated embodiment, the manifold sleeve 260 is constructed to slide onto the distal ends of the evacuation tube 112 and pressure tube 110. In this particular embodiment, the manifold 200 is formed of at least two components, the sleeve 260 and the nozzle end 250 which may be welded together. It should be appreciated that in other embodiments, the sleeve 260 may be formed integrally with the nozzle end 250, and that the manifold 200 may be coupled to the pressure tube 110 and/or the evacuation tube 112 differently. It should also be appreciated that in other embodiments, the distal tips of the tubes 110, 112 may be coupled in a variety of other ways, and/or the tubes may be coupled together at a location(s) other than or in addition to their distal tips, or the tubes may not be coupled together at all, as the invention is not limited in this respect. FIG. 4 illustrates another embodiment of a manifold 200 which is coupled to the distal tip of a pressure tube 110. The particular manifold 200 illustrated in FIG. 4 is not configured to be coupled also to the evacuation tube 112. In one embodiment, the manifold 200 is manufactured from a block of material, such as stainless steel. The manifold 200 may include a first lumen 402 to couple the pressure tube 110 to the manifold and a second lumen 404 may be created at an angle transverse to the pressure tube 110 to fluidically connect the pressure tube 110 to a nozzle 116. In one embodiment, the second lumen 404 is formed by making a bore in the manifold 200 through one end 406 and thereafter closing the end 406, for example with a welded bead 405. The lumens and nozzle openings in the manifold may be formed in a variety of ways known in the art of machining, such as by drilling or electro-discharge machine (EDM) cutting. In one embodiment, the diameter of the nozzle 116 is between approximately 0.003 inches-0.008 inches (0.075 mm-0.2 mm). In another embodiment, the diameter of the nozzle is between approximately 0.004 inches-0.0055 inches (0.1 mm-0.175 mm). In another embodiment, the nozzle may comprise a nozzle insert made of the same or a different material as manifold 200. For example, in one such embodiment, the nozzle is formed from a hard metal disk, a ceramic, glass or similar non-metallic tubular insert with an orifice that is affixed within an opening the manifold 41 by, for example, swaging a collar surrounding the disk, gluing, soldering or welding. In the embodiment of FIG. 4, portions of the distal end of the evacuation tube 112 may be coupled to the pressure tube 110, such as by bonding or fasteners. Other types of manifolds contemplated are discussed in co-pending Provisional Application No. 60/794,867, which is herein incorporated by reference in its entirety.

In certain embodiments, as the distal ends of the pressure and evacuation tubes 110, 112 are altered from a first configuration, in which the distal ends are retracted within the sheath 114, to a second deployed configuration which may, for example, be similar to the configurations illustrated in FIGS. 2a-2b or 2c, it may be important to maintain or bring the nozzle 116 into alignment with the jet-receiving opening 118 and to keep a consistent distance separating the nozzle from the jet receiving opening. Alignment and consistent spacing of the nozzle 116 and the jet-receiving opening 118 keeps the liquid jet 120 emitted from the nozzle 116 from becoming misdirected and missing or partially missing the jet-receiving opening 118 target or the liquid jet length from changing undesirably. However, as the distal end of the instrument is deployed from a first, stowed configuration to the second, deployed configuration, the change in the radius of curvature of the pressure lumen 110 may be less than the change in the radius of curvature of the evacuation lumen 112, since the arc length of the pressure lumen will be greater than that of the evacuation lumen in the second configuration. Of course, for other embodiments having other deployed configurations, the situation may be reversed or the radii of curvature and arc lengths of the two tubes may not change relative to each other (see discussion below). For example, as shown in FIG. 3a, in one particular configuration, the radius of curvature R_{P} of the pressure tube 110 is greater than the radius of curvature R_{E} of the evacuation tube 112. This difference in the radius of curvature may make it difficult to maintain alignment and consistent spacing between the nozzle 116 and the jet-receiving opening 118 for tubes of fixed length rigidly coupled together because the pressure lumen 110 will bend slightly less than the evacuation lumen 112 upon deployment and the length of the distal portion of the pressure tube will need to become somewhat greater than that of the evacuation tube. It will tend to be even more difficult to maintain alignment and spacing of the nozzle 116 and the jet-receiving opening 118 if the difference between the arc length of the distal end shapes in the first and second configurations are even more dramatic than illustrated.

One approach to maintaining or bringing the nozzle 116 in alignment with the jet-receiving opening 118 and/or maintaining a consistent jet length upon deployment is to make portions of either or both of the pressure tube and the evacuation tube extendible. For example, when the pressure tube 10 is extendible, as the configuration of the distal ends of both the pressure and evacuation tubes 110, 112 are selectively controlled and deployed, portions of the pressure tube 110 may extend to maintain facilitate the needed increase in the arc length of the pressure tube relative to that of the evacuation tube. In one embodiment, as illustrated in FIG. 2a, the pressure tube 110 includes a coiled extendable section 211 providing the necessary slack to make up the change in arc length upon deployment and straightening of the distal end of the instrument. In this particular illustrated embodiment, the coiled section 211 is contained within the body 104 of the instrument 102. In the embodiment illustrated in FIG. 2a, the coiled section 211 is helical shaped, but in other embodiments, the coiled section 211 may include one or more bends and/or stretchable portions in the tube 5 such that the tube is extendible or any other means of providing excess length (i.e. slack).

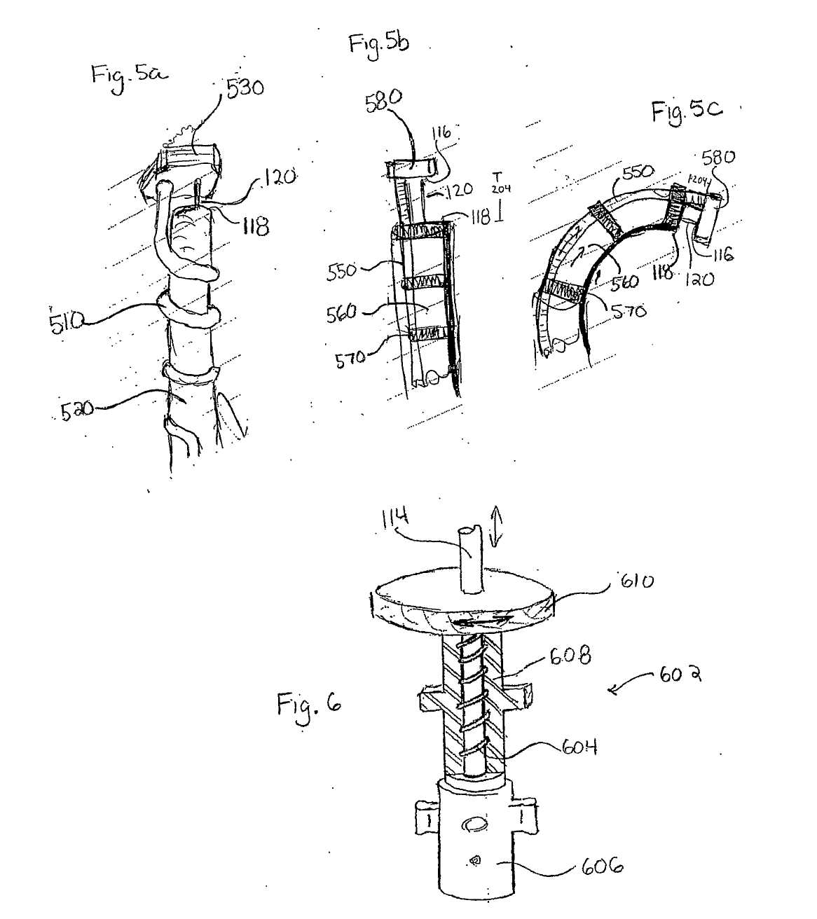

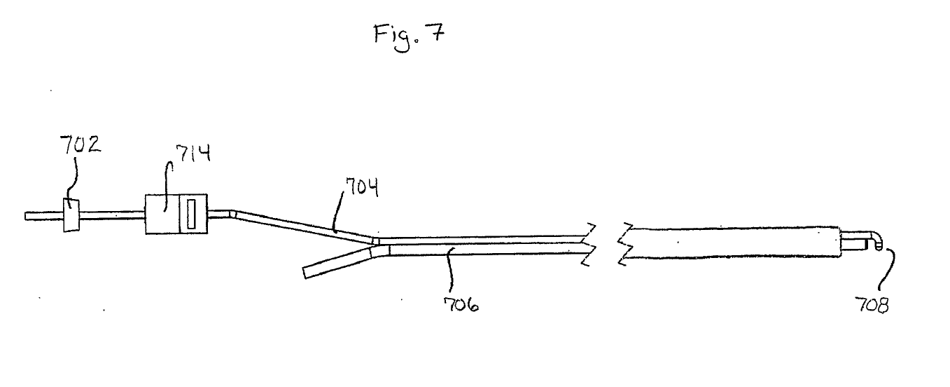

In another embodiment, the pressure tube 110 may be made extendible by providing a coiled section which may extend outside of the handpiece body. For example, as illustrated in FIG. 5a, a coiled section 510 of the pressure tube may wrap around the evacuation tube 520. In the illustrated embodiment, substantially all of the distal end of the pressure tube is coiled, but it should be appreciated that in other embodiments, only lesser portions of the distal end of the pressure tube may form a coiled section 510 around the evacuation tube 520. As shown, a manifold 530 may be coupled to the pressure tube and the nozzle 116 may be formed in the manifold to direct a liquid jet 120 towards the jet-receiving opening 118 of the evacuation tube 520. To maintain a consistent jet length during deployment, the pressure tube may be rigidly coupled to the evacuation tube at at least one location at the distal end distal to at least a portion of the coiled portion of the pressure tube.

In another embodiment, illustrated in FIGS. 5b-5c, the distal end of the pressure tube 550 is movable relative to the distal end of the evacuation tube 560 such that the nozzle 116 may maintain alignment with respect to the jet-receiving opening 118 as the tubes bend, while the jet length 204 is permitted to change as the tubes bend. In this particular embodiment, the pressure tube 550 is slidably coupled to the evacuation tube 560 by bands 570 which are spaced apart along the distal end of the tubes. A manifold 580 is coupled to the pressure tube 550 and the nozzle 116 is formed in the manifold 580. As the shape of the distal end of this instrument is altered from the first configuration in FIG. 5b to the second configuration shown in FIG. 5c, the distance 204 between the nozzle 116 and the jet-receiving opening 118 lessens to compensate for the different in the radius of curvature and arc length. In one embodiment, the amount in which the distance 204 between the nozzle 116 and the jet-receiving opening 118 lessens is approximately equal to one diameter of the evacuation tube 560 when the tube is bent through an arc of about 90 degrees.

It should also be recognized that in other embodiments, the evacuation tube as opposed to or in addition to the pressure tube may be extendible such that as the configuration of the distal ends of the both the pressure and evacuation tubes are selectively controlled, the nozzle 116 maintains or is brought into alignment with the jet-receiving opening 118 and, in certain embodiments, the jet length remains relatively constant. For an embodiment where only the evacuation tube is extendible, the distal ends of the pressure and evacuation tubes during deployment may bend in a substantially opposite direction as that shown in FIGS. 2a-2b (i.e. bending to the left side of the figure rather than the right side), such that the difference in the radius of curvature of the evacuation lumen 112 is slightly less than the difference in the radius of curvature of the pressure lumen 110. It is contemplated that the pressure tube 112 may be extendible in any of the ways in which the pressure tube 110 may be extendible as discussed above.

As shown in FIGS. 3a-3b, the surgical instrument may also include an aligner 370 constructed and positioned to maintain the position of the pressure tube 110 relative to the evacuation tube 112. In this illustrative embodiment, the aligner 370 is positioned within the tubular sheath 114, is coupled to either pressure tube 110 or the evacuation tube 112 and slidably receives at least a portion of the other (i.e. the one to which it is not coupled). The aligner 370 may be used to maintain a substantially parallel alignment of the pressure tube 110 and the evacuation tube 112 inside of the sheath 114 to minimize friction during the relative movement of the sheath 114 and the tubes. In the view shown in FIG. 3b, the aligner 370 is coupled to the evacuation tube 112. In this particular embodiment, the aligner 370 comprises two back to back “C” shaped segments 375, 377 which cradle the tubes 110 and 112, respectively. A figure-eight shaped aligner (not shown) may also be used in embodiments where there is sufficient space inside the sheath 114. It is also contemplated that in some embodiments, the aligner may be integrally formed with the sheath. In one particular embodiment, the length of the aligner is approximately 12 mm.

In some of the above described embodiments, the pressure tube 110 is maintained substantially parallel to the evacuation tube 112. In other embodiments, it is also contemplated that at least portions of the pressure tube 110 may be contained within the evacuation tube 112. In one embodiment, at least certain portions of the pressure tube are contained within the evacuation tube; however the distal end of the pressure tube extends away from the distal tip of the evacuation tube so that the nozzle is be positioned opposite the jet-receiving opening at a desired separation distance. In one embodiment, the longitudinal axis of the pressure tube may be substantially coaxial with the longitudinal axis of the evacuation tube. One advantage of a substantially coaxial arrangement is that as the tubes are curved and straightened during deployment and retraction, the difference between the radii of curvature and arc length of these two tubes may be minimized or essentially eliminated reducing the need to provide slack to either tube to maintain alignment or jet length. An embodiment where a distal end portion of the pressure tube is contained within and is substantially coaxial with the evacuation tube may help to maintain the jet length and/or alignment between the nozzle and the jet-receiving opening as the shape of the distal end of the instrument changes during deployment and retraction.

FIG. 2d illustrates yet another embodiment of the distal end of a liquid jet surgical instrument according to the present invention. As shown, in this embodiment, the distal ends of the pressure and evacuation tubes 110, 112 are bent in a different plane than the embodiments shown in FIGS. 2a-2c, such that the radius of curvature and arc length of the evacuation tube 112 is substantially equal to the radius of curvature and arc length of the pressure tube 110 as the arc length of the two tubes changes upon deployment. The configuration illustrated in FIG. 2d would be similar to an embodiment in which the distal end of the instrument illustrated in FIG. 2a was bent out of or into the page. In this embodiment, the nozzle 3 may more easily maintain alignment with the jet-receiving opening 38.

The particular configuration and shape of the distal end of the pressure tube and evacuation tube in a second, deployed configuration may vary according to different embodiments of the present invention. In one embodiment, the distal end curvature of the instrument shown in FIGS. 2a-2b has reached an essentially completely relaxed state. In this embodiment, further relative movement of the sheath 114 proximally with respect to the tubes 110, 112 may not substantially further alter the shape of the distal end of the tubes 110, 112. In one embodiment, the distal end of the pressure tube 110 in a second, deployed configuration has a bent configuration such that an angle between a longitudinal axis of the proximal end of the surgical instrument and the center line of the nozzle is at least about 180 degrees (e.g. see FIG. 2a-2b where the angle is about 180 degrees). In another embodiment, the distal end of the pressure tube in the second configuration has a bent configuration such that an angle between a longitudinal axis of the proximal end of the surgical instrument and the center line of the nozzle is at least about 90 degrees. FIG. 2c illustrates an embodiment where in the second configuration the angle between a longitudinal axis of the proximal end of the surgical instrument and the center line of the nozzle is about 90 degrees. Tubes 110 and 112, of instrument 102 in the configuration shown in FIG. 2c are in a relatively relaxed state (i.e. relative to their essentially straight configuration when sheath 114 is slid fully proximally) but are not essentially fully relaxed as illustrated in FIG. 2b. Although not explicitly illustrated, it should be appreciated that in yet other embodiments, the distal end of the pressure tube in a second configuration has a bent configuration such that an angle between a longitudinal axis of the proximal end of the surgical instrument and the center line of the nozzle is at least about 45 degrees in one embodiment, and is at least about 10 degrees in another embodiment.

Although the above described embodiments are directed to configurations where the straightener at least partially surrounds the pressure and evacuation tubes 110, 112, the invention is not limited in this respect. Embodiments where the straightener extends within or adjacent to at least one of the pressure and evacuation tubes are also contemplated by the present invention. For example, in one embodiment, instead of partially enclosing the tubes 110, 112 the straightener(s) may be contained within or adjacent to one or both of the tubes and be movable/slidable with respect to the tubes to effect deployment/straightening. It is contemplated that in some embodiments, one or more straighteners may be contained and extend within both the pressure and evacuation tubes. In other embodiments, the straightener may extend adjacent to both the tubes. In one embodiment, when the straightener extends and is contained within or is adjacent to one or both of the pressure and evacuation tubes the straightener may be slidable with respect to the tubes. Whereas in another embodiment, the pressure and evacuation tubes are slidable with respect to the straightener to effect a change in the shape of the distal end of both the pressure and evacuation tubes.

The surgical instruments in certain embodiments may be configured so that the relative movement between the straightener and the pressure and evacuation tubes may be controlled by an operator of the instrument. In one embodiment, the operator may manually move either the straightener or the tubes to cause the relative movement. The straightener and/or the tubes may include a deployer to facilitate control of the deployment movement by an operator. Many potential deployer configurations to facilitate this control will be readily apparent to the skilled artisan and are within the scope of the present invention. In an exemplary embodiment, the deployer may comprise a gripping region, collar or knob attached to the sheath to help facilitate this manual relative movement. For example, as shown in FIG. 2a, the straightener is a sheath 114 which includes a deployer comprising a collar with a gripping region 216 which projects out from the sheath 114 and is attached thereto. An operator may move the sheath by holding region 216 and moving the deployer either distally or proximally. As the deployer 216 is moved, the sheath 114 slides through a bearing 209 in the handpiece body 104. Markings may be provided on the straightener and/or the tubes to provide an indication of the relative position. In one embodiment, the sheath 114 may include a plurality of discrete telescoping segments having a known length, where retraction of the sheath by a certain number of segments may translate into a predetermined distal configuration. In some embodiments, the movement of the straightener relative to the pressure and evacuation tubes 110, 112 may be controlled, such that the angular orientation and/or radius of curvature of the distal end of the pressure and evacuation tubes may be known based upon the amount of relative movement between the straightener and the tubes.

In another embodiment, a deployer may be operatively connected to at least one of the straightener and the pressure and evacuation tubes and controllable by an operator of the instrument to control movement of the straightener relative to the pressure and evacuation tubes. One type of deployer is illustrated in FIG. 6 and includes a threaded mechanism 602. In this embodiment, a hollow male screw thread 604 is coupled to a portion 606 of the instrument 102 and the sheath 114 passes through the screw thread 604. A mating female threaded tube 608 may attach directly or indirectly (such as through a bearing) to the sheath 114. The female threaded tube 608 may be manually rotated by a thumbwheel 610 to cause the female threaded tube 608 to rotate and move in a longitudinal direction. This longitudinal movement of the female threaded tube 608 causes longitudinal movement of the sheath 114 to facilitate control of the relative movement between the sheath 114 and the pressure and evacuation tubes.

It should be appreciated that in other embodiments the deployer may be configured differently, as the invention is not limited in this respect. For example, other known threaded mechanisms may be used to control the relative movement between the sheath and the pressure and evacuation tubes. In other embodiments, a rack and pinion gear may be used as a deployer. Rotation of a thumbwheel 610 may rotate a pinion gear (not shown) to cause the rack (not shown) and sheath 114 to move longitudinally. In another embodiment, the deployer includes a lever (not shown). Various other types of known deployer mechanisms may be used to control the relative movement of the straightener and the pressure and evacuation tubes.

In some embodiments, one or more surfaces of the distal end of the surgical instrument may be constructed to be tissue cutting surfaces, e.g. by providing a sharpened cutting edge. In one embodiment, surfaces of the manifold 200, pressure tube 110, and or evacuation tube 112 may include cutting surfaces to provide mechanical tissue removal. Tissue removed by these cutting surfaces may be evacuated by the liquid jet through the evacuation lumen 112 in certain embodiments. A variety of designs for such scraping/cutting surfaces are described in co-pending US. Published Application No. 2004-0243157 A1 which is incorporated herein by reference.

It is also contemplated that in some embodiments where the pressure tube is extendable to maintain or bring the nozzle into alignment with the jet-receiving opening or to maintain a consistent jet length, the pressure tube may only be selectively extendable. In other words, the operator may control when the pressure tube is capable of being extended. In one embodiment, a sealing gasket 702 such as that described in commonly owned U.S. Pat. No. 6,923,792 which is herein incorporated by reference, is used at the proximal end of the pressure tube 704 to selectively control the extendibility of the pressure tube. A schematic illustration of a surgical liquid jet system including a sealing gasket 702 is shown in FIG. 7. The system includes a pressure tube 704 and an adjacent evacuation tube 706 with a distal end 708 as discussed above. In this particular embodiment, the distal end of the evacuation tube 706 and pressure tube 704 extend within sheath 714 in a substantially straight non-relaxed, undeployed configuration. The sealing gasket 702 is positioned at the proximal end of the pressure tube 704 such that the high pressure fluid seals the gasket 702 to the pressure tube 704. In one embodiment, the sealing gasket 702 may be, for example, fixed to the handpiece body (not shown). In one embodiment, the sealing gasket 702 is positioned downstream of a filter 714 and may enable an operator to selectively control the length of the pressure tube 704. As discussed in greater detail in the '792 patent, when the a high pressure fluid flows through the pressure tube 704, the high pressure fluid creates a tight seal around the gasket 702 such that the pressure tube 704 is not moveable with respect to the sealing gasket 702. In the absence of a high pressure, fluid flowing through the pressure tube 704, the pressure tube 704 is slidable with respect to the gasket 702. In embodiments where the pressure tube 704 is extendable, the sealing gasket 702 may be selectively used to extend a portion of the pressure tube if needed. In this embodiment, the sealing gasket 702 is constructed such that the pressure tube 704 is extendable only when there is no high pressure fluid flowing through the pressure tube 704. Thus, an operator can shut off the high pressure fluid supply into the pressure tube 704 so that she can change the shape of the distal end of the instrument, as discussed above. It should be appreciated that similarly, the evacuation tube may be configured to be similarly selectively extendable as the invention is not so limited. It is also contemplated that in other embodiments, a sealing gasket which permits movement of the pressure tube while under fluid pressure may also be employed.

The separation distance 120 between the nozzle and the jet-receiving opening depends upon the requirements of the particular surgical procedure for which the surgical instrument is used; however, for some typical embodiments, the distance will have a maximum value of about 1 cm, for other typical embodiments, between about 2-6 mm, and for yet other typical embodiments, about 3 mm. The jet-receiving opening 118 may have a diameter of between about 0.01 and about 0.2 inches, in other embodiments between about 0.03 and about 0.1 inches, and in some embodiments a diameter of about 0.06 inches.

In certain embodiments of the invention, a surgical liquid-jet instrument is employed for use in a surgical method. The distal end of a surgical liquid jet instrument is inserted into a surgical site in the body of a patient. Relative movement between a straightener and a pressure tube and an evacuation tube of the surgical liquid-jet instrument is created such that at least the distal ends of both the pressure and evacuation tubes undergo bending as the straightener and the pressure and evacuation lumen are moved relative to each other. One or more of the radius of curvature of the distal ends of both the pressure and evacuation tubes, the arc length of a curved portion of the distal ends of both the pressure and evacuation tubes, and the angular orientation of the center lines of both a nozzle in fluid communication with the pressure tube and a jet-receiving opening of the evacuation tube with respect to a longitudinal axis of the proximal end of the instrument changes as the tubes bend. A liquid jet is created with the surgical liquid-jet instrument by flowing a liquid under high pressure through the nozzle in fluid communication with the pressure tube. The liquid jet is directed towards the jet-receiving opening of the evacuation tube of the surgical liquid-jet instrument and selected tissue within the surgical site is cut or ablated with the liquid jet.

In certain embodiments of the invention, an inventive surgical liquid-jet instrument is employed for use in a surgical method, where the distal end of the instrument is inserted into a surgical site in the body of a patient in a firsts configuration. The distal end of the surgical liquid-jet instrument is deployed into a second configuration. The distal ends of a pressure tube and evacuation tube of the surgical liquid-jet instrument undergo bending or straightening as the instrument is deployed into the second configuration. The shape of the distal end of the pressure and evacuation tubes is specifically adapted for the particular surgical site when in the deployed second configuration. A liquid jet is created with the surgical liquid-jet instrument by flowing a liquid under high pressure through the nozzle in fluid communication with the pressure tube. The liquid jet is directed towards the jet-receiving opening of the evacuation tube of the surgical liquid-jet instrument and selected tissue within the surgical site is cut or ablated with the liquid jet.

In certain embodiments, the above described surgical liquid-jet instrument and surgical methods may be used for surgery of the spine, brain, prostate, bladder, breast, heart, nasal sinuses, liver, lungs, various joints, gall bladder, kidneys, ovaries, and other organs having a confined space.

One aspect of the invention involves the discovery that certain problems may arise when certain conventional surgical liquid jet instruments are used in surgical procedures, especially in a confined space within the body. For example, when an instrument is inserted into a confined body space, the instrument may not be configured to be able to maneuver well within the confined space. The dimensions of the components at the distal end of the instrument may be selected to be small to enable the instrument to fit into the confined space. However, once in the confined space, the configuration of the distal end of the instrument may not be as suitable for performing a particular surgical procedure in the confined space.

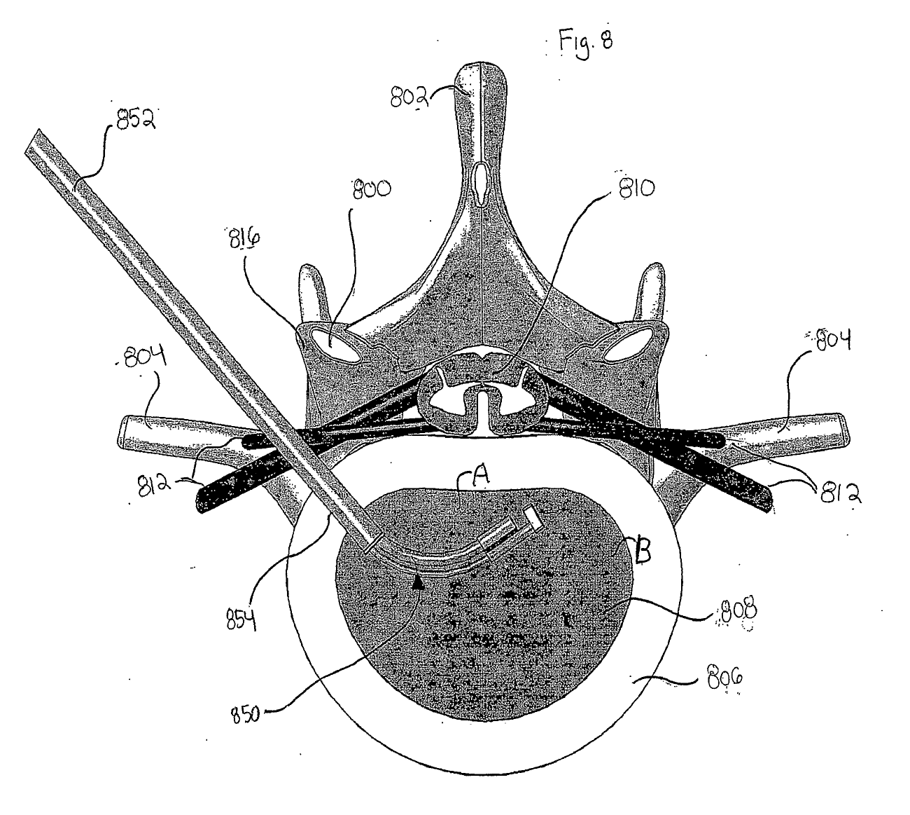

As shown in FIG. 8, the inventive surgical liquid jet instruments where the distal ends of both the pressure tubes and evacuation tubes have more than one configuration may be well suited for insertion into the spine of a patient for spinal surgery applications. The spinal column is made up of the vertebrae bones which are connected in the anterior (front) portion of the spine by intervertebral discs. The intervertebral discs provide support and cushioning to the spine, serving as the spine's shock absorbing system. The facets 800 are where one vertebrae contacts another vertebrae. The discs also allow for some spinal motion, although individual disc movement is very limited. Many ligaments and muscles are also attached to the posterior (back) portion of the spine to provide power for spine movement. The spinous process 802 and the transverse processes 804 serve as anchors for the ligaments. Each intervertebral disc is composed of an outer ring-like component made up of concentric sheets of collagen fibers, called the annulus fibrosus 806, and an inner semi-gelatinous tissue, called the nucleus pulposus 808. The radial structure of the annulus fibrosus 806 prevents the nucleus pulposus 808 from protruding from the disc. In the spinal column, there are four segments of spinal curvatures. From the superior (top) to the inferior (bottom) portions of the spinal column, these curvatures include the cervical, thoracic, lumbar, and sacral portions.

Surgical procedures on intervertebral discs in the lumbar, cervical, or thoracic portions of the spine are performed for a variety of reasons, which include treatment of tears in and herniation or rupture of the annulus fibrosus 806, herniation or loss of the nucleus pulposus 808, and significant disc height loss. Herniation results when the annulus fibrosus 806 weakens such that the soft central nucleus putposus 808 bulges through the layers of the annulus fibrosus 806. The nucleus pulposus 808 may bulge or leak posteriorly towards the spinal cord 810 and major nerve roots 812, causing significant pain and discomfort.

One of the most common surgical procedures for treating a disc herniation is a discectomy. This procedure involves the removal of portions of the disc which impinge on the nerve roots 812 or spinal cord 810 posterior to the disc. All or portions of the nucleus pulposus 808 may be removed to minimize the risk of additional herniations. The nucleus pulposus 808 may be accessed by a variety of recognized surgical techniques. In certain embodiments, the nucleus pulposus 808 is accessed directly through the annulus fibrosus 806. For example, the nucleus pulposus may be accessed by an incision through either the anterior portion or the posterior portion of the annulus fibrosus 806. In other embodiments, where an opening has already formed within the annulus fibrosus, it may be desirable to access the nucleus pulposus through this opening. In yet other embodiments, the nucleus pulposus is accessed via the vertebral body or through an end plate. For example, in certain embodiments, the nucleus pulposus may be accessed by penetrating into the spinal column through the sacral portion. It should be appreciated that in certain embodiments, the inventive surgical instruments may be inserted into the spine using a variety of techniques known for entering the spine, as would be recognized by one skilled in the art.

Various devices may be used to replace portions of the removed nucleus pulposus and/or annulus fibrosus, or the disc entirely. For example, when only the nucleus pulposus 808 is replaced, a prosthetic device may be inserted through a hole created in the annulus fibrosus 806. Once the prosthetic device is within the confines of the annulus fibrosus 806, the device may expand, inflate, or deploy to fill the area of the disc that was removed.

In certain surgical applications, it may be desirable to remove all or portions of the inner nucleus pulposus 808, leaving the annulus fibrosus 806 as intact as possible. However, conventional surgical instruments that are used to remove portions of the intervertebral disc may not be capable of maneuvering within the annulus fibrosus 806 to be able to access and remove the appropriate portions of the inner nucleus pulposus 808. For example, in the confined surgical site shown in FIG. 8, it may be desirable for the instrument to access portions of the nucleus pulposus 808 near the spinal cord 810 at region B, as well as on another side of the disc at region B.

As mentioned previously, because the inventive surgical liquid jet instruments can be configured and operated to provide a plurality of distal end configurations with a geometry and contour (or range of geometries and contours over a range of deployment) specifically designed for a specific surgical site(s), they may, according to certain embodiments of the invention wherein the instruments are specifically configured for surgical procedures within the spine, be advantageously utilized in surgical procedures to remove all or portions of the inner nucleus pulposus. Because the shape of the distal end of the instrument may be changed when the instrument is already deployed into the surgical site, the instrument may be particularly well suited to remove portions of the nucleus pulposus, for example, at region A and B. The instrument may be configured and deployed so as to leave other portions of the nucleus pulposus, and/or the annulus fibrosus, and/or other portions of the spine, such as the cartilage of the end plates, as intact as possible.

For example, as illustrated in FIG. 8, an instrument 850, which may include a cannula 852 may be inserted through an opening 854 in the annulus fibrosus 806 in a first configuration, which may have a substantially straight distal end. The opening 854 through which a surgical instrument may be inserted may be only approximately 1 cm in diameter, or in another embodiment, approximately 0.5 cm in diameter. The cannula should be inserted into the spine in a manner to avoid the spinal cord 810, the facets 800, the pedicle 816, the spinous process 802 and the transverse processes 804. Thereafter, the shape of the distal end may be changed into a second bent configuration, as illustrated in FIG. 8. To access other portions in the surgical site, the shape of the distal end of the instrument 850 may be further adjusted to a third configuration. It should be appreciated that in some embodiments, the distal end of the instrument is configured to adjust its shape to follow the inner contour of the annulus fibrosus 806 of an intervertbral disc.

In certain embodiments, such as for those instruments specifically designed for spinal applications, the outer diameter of the evacuation tube may range from about 0-5 mm-about 2 mm, and the outer diameter of distal end of the instrument, including the evacuation tube combined with the pressure tube may range from about 0.8 mm-about 3 mm. Upon insertion of the instrument into the disc, the operator of the instrument can then deploy the distal end of the instrument into a different configuration. The operator can then turn on a pump or dispenser supplying high pressure liquid to the device, as discussed previously, in order to create a liquid jet with the surgical instrument. The liquid jet can then be directed towards the jet receiving opening of the evacuation tube of the instrument, which can be effective for cutting or ablating a selected tissue within the intervertebral disc.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention. All definitions, as defined and used herein, should be understood to control over dictionary definitions, definitions or usage in documents incorporated by reference, and/or ordinary meanings of the defined terms.

It should also be understood that, unless clearly indicated to the contrary, in any methods claimed herein that include more than one step or act, the order of the steps or acts of the method is not necessarily limited to the order in which the steps or acts of the method are recited.

In the claims (as well as in the specification above), all transitional phrases or phrases of inclusion, such as “comprising,” “including,” “carrying,” “having,” “containing,” “composed of,” “made of,” “formed of” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

What is claimed is:

1. A surgical instrument comprising:
a distal end adapted to perform a surgical procedure on a patient and a proximal end;
a pressure tube having sufficient burst strength to conduct a high pressure liquid towards the distal end of the instrument, the pressure tube including at least one nozzle providing a jet opening, the nozzle being shaped to form a liquid jet as the high pressure liquid flows therethrough;
an evacuation tube including a jet-receiving opening locatable opposite the jet opening, wherein the nozzle is aligned with the jet-receiving opening to receive the liquid jet when the instrument is in operation;
wherein at least the distal ends of both the pressure and evacuation tubes have a first configuration in a non-relaxed state and a second configuration in a more relaxed state; and
a straightener constructed and arranged to selectively control the configuration of the distal ends of both the pressure and evacuation tubes to change one or more of the radius of curvature of the distal ends of both the pressure and evacuation tubes, the arc length of a curved portion of the distal ends of both the pressure and evacuation tubes, and the angular orientation with respect to a longitudinal axis of the proximal end of the surgical instrument of both a center line of the nozzle and a center line of the jet-receiving opening.

2-6. (canceled)

7. A surgical instrument as in claim 1, wherein the straightener comprises a tubular sheath which surrounds at least a portion of the pressure tube and the evacuation tube, and wherein at least one of the sheath and the pressure and evacuation tubes are slidable with respect to the other to effect a change in the shape of the distal ends of both the pressure tube and the evacuation tube.

8. A surgical instrument as in claim 7, wherein the tubular sheath comprises a cannula through which the surgical instrument is introduced into the body of the patient.

9. A surgical instrument as in claim 1, wherein the sheath is slidable with respect to the pressure and evacuation tubes.

10-13. (canceled)

14. A surgical instrument as in claim 1, further comprising a handpiece having a body, wherein the proximal end of the pressure tube and the evacuation tube pass through the body.

15. A surgical instrument as in claim 1, further comprising a deployer operatively connected to at least one of the straightener and the pressure and evacuation tubes and controllable by an operator of the instrument, wherein the deployer controls the movement of the straightener relative to the pressure tube and evacuation tube.

16. (canceled)

17. (canceled)

18. A surgical instrument as in claim 1, wherein at least a portion of the distal end of the pressure tube is coupled to the distal end of the evacuation tube to prevent relative motion therebetween.

19. A surgical instrument as in claim 18, wherein the pressure lumen further comprises a manifold, wherein the nozzle is formed in the manifold.

20-23. (canceled)

24. A surgical instrument as in claim 8, further comprising an aligner positioned within the sheath, wherein the aligner is coupled to one of the pressure tube and the evacuation tube and slidably receives at least a portion of the other of the pressure tube and the evacuation tube.

25. A surgical instrument as in claim 1, wherein the high pressure liquid is supplied to the jet opening when the instrument is in operation at a pressure of at least 1,000 psig.

26. (canceled)

27. A surgical instrument as in claim 1, wherein the distal end of the surgical instrument has a shape and size specifically configured to perform a surgical procedure in a predetermined region of the body of the patient defining a surgical site.

28-33. (canceled)

34. A surgical instrument as in claim 15, wherein the evacuation lumen is shaped and positionable to enable evacuation of essentially all of the liquid comprising the liquid jet from the jet-receiving opening to the proximal end of the instrument, without the need for an external source of suction.

35. A method comprising:
inserting at least a portion of a surgical liquid-jet instrument comprising a distal end adapted to perform a surgical procedure on a patient and a proximal end into a surgical site in the body of a patient;
creating relative motion between a straightener and a pressure tube and an evacuation tube of the surgical liquid-jet instrument, wherein at least the distal ends of both the pressure and evacuation tubes undergo bending or straightening as the straightener and the pressure and evacuation tubes are moved relative to each other such that one or more of the radius of curvature of the distal ends of both the pressure and evacuation tubes, the arc length of a curved portion of the distal ends of both the pressure and evacuation tubes, and the angular orientation of the center lines of both a nozzle in fluid communication with the pressure tube and a jet-receiving opening of the evacuation tube with respect to a longitudinal axis of the proximal end of the surgical instrument changes as the tubes bend;
creating a liquid jet with the surgical liquid-jet instrument by flowing a liquid under high pressure through the nozzle in fluid communication with the pressure tube;
directing the liquid jet towards the jet-receiving opening of the evacuation tube of the surgical liquid-jet instrument; and
cutting or ablating a selected tissue within the surgical site with the liquid jet.

36. A method as in claim 35, further comprising: distally moving the straightener relative to the pressure and evacuation tubes to straighten the distal ends of both the pressure tube and the evacuation tube.

37. A method as in claim 35, further comprising: proximally moving the straightener relative to the pressure and evacuation tubes to increase the degree of bending of the distal ends of both the pressure tube and the evacuation tube.

38-40. (canceled)

41. A method comprising:
inserting at least a portion of a surgical liquid-jet instrument comprising a distal end adapted to perform a surgical procedure on a patient and a proximal end into a surgical site in the body of a patient, wherein the distal end of the surgical liquid-jet instrument is in a first configuration as it is inserted into the surgical site;
deploying the distal end of the surgical liquid-jet instrument into a second configuration, wherein the distal end of the surgical liquid-jet instrument comprises a pressure tube and an evacuation tube which undergo bending or straightening as the instrument is deployed into the second configuration, wherein the shape of the distal end of the pressure and evacuation tubes is specifically adapted for the surgical site when in the deployed configuration; creating a liquid jet with the surgical liquid-jet instrument by flowing a liquid under high pressure through a nozzle in fluid communication with the pressure tube;
directing the liquid jet towards a jet-receiving opening of the evacuation tube of the surgical liquid-jet instrument; and
cutting or ablating a selected tissue within the surgical site with the liquid jet.

42. A method as in claim 41, wherein the act of deploying the distal end of the surgical liquid-jet instrument into the second configuration comprises: creating relative motion between a straightener and the pressure and evacuation tubes such that the pressure and evacuation tubes undergo bending or straightening as the straightener and the pressure and evacuation tubes are moved relative to each other so that the angular orientation of both a center line of the nozzle and a center line of the jet-receiving opening with respect to the longitudinal axis of the proximal end of the surgical instrument changes as the tubes bend or straighten.

43. A method as in claim 42, further comprising: distally moving the straightener relative to the pressure and evacuation tubes to straighten the distal ends of both the pressure tube and the evacuation tube.

44. A method as in claim 42, further comprising: proximally moving the straightener relative to the pressure and evacuation tubes to increase the degree of bending of the distal ends of both the pressure tube and the evacuation tube.

45. (canceled)

46. A method of manufacturing a surgical liquid-jet instrument comprising a pressure tube and an evacuation tube, the method comprising:
forming a bend in the distal end of a pressure tube of the surgical liquid-jet instrument, wherein the pressure tube has sufficient burst strength to conduct a high pressure liquid towards a distal end of the instrument, the pressure tube including at least one nozzle providing a jet-opening, wherein the nozzle is shaped to form a liquid jet as a liquid at high pressure flows therethrough; forming a bend in the distal end of an evacuation tube of the surgical liquid-jet instrument, wherein the evacuation tube includes a jet-receiving opening having a cross-sectional area located opposite the jet-opening;
slidably connecting a straightener to at least the distal ends of both the pressure and evacuation tubes, wherein the straightener is constructed and arranged to selectively control the configuration of the distal ends of both the pressure and evacuation tubes to change one or more of the radius of curvature of the distal ends of both the pressure and evacuation tubes, the arc length of a curved portion of the distal ends of both the pressure and evacuation tubes, and the angular orientation with respect to a longitudinal axis of the proximal end of the surgical instrument of both a center line of the nozzle and a center line of the jet-receiving opening.

47. A method as in claim 46, wherein the straightener comprises a sheath surrounding at least the distal ends of both the pressure and evacuation tubes.

48. A method as in claim 46, wherein bends in the distal ends of both the pressure tube and evacuation tubes created during the forming acts are formed by heating the distal ends to a temperature of at least approximately 7500 F and thereafter quenching the distal ends in a cooling fluid.
